(19) United States
(12) Patent Application Publication
(10) Pub. No.: US 2008/0108880 A1
(43) Pub. Date: May 8, 2008

(54) Wireless transmission apparatus for transmitting physiological parameters and method thereof

(75) Inventors: Shuenn-Tsong Young, Taipei City (TW); Chung-Wang Lee, Hsinchu (TW); Tsair Kao, Taipei City (JP); Chang-Yu Liu, Yuanlin Township (TW)

Correspondence Address: BACON & THOMAS, PLLC, 625 SLATERS LANE, FOURTH FLOOR, ALEXANDRIA, VA, 22314, US

(73) Assignee: National Yang-Ming University, Taipei City (TW)

(21) Appl. No.: 11/592,190

(22) Filed: Nov. 3, 2006

(51) Int. Cl. A61B 5/00

(52) U.S. Cl. 600/300

(57) ABSTRACT

A wireless transmission apparatus includes a processing unit, a pushbutton for producing a press signal, a memory unit for storing an identification code, a wireless transmission unit, and a connecting unit connected to a portable detector for receiving a physiological parameter measured by the portable detector. If the connecting unit is not connected and the press signal is received, the processing unit will produce and send an emergency notice signal to the wireless transmission unit that packs the emergency notice signal and identification code as an emergency notice packet to be sent to a service provider. If the connecting unit is connected and the press signal is received, the processing unit will output the physiological parameter to the wireless transmission unit for packing the physiological parameter and identification code as a physiological data packet to be sent to a service provider for monitoring the user's current physiological conditions.

### FIELD OF THE INVENTION

The present invention relates to a wireless transmission apparatus for transmitting physiological parameters and its method, and more particularly to a wireless transmission apparatus for transmitting physiological parameters and its method capable of expanding the scope of living activities of a user and eliminating the dead corner of remote homecare.

### BACKGROUND OF THE INVENTION

As current living standard and medical technology advance, our average life become increasingly longer, and thus the percentage of the ageing population gradually climbs up, and thus various different issues including social welfare, medial treatment and technology and social security system are emerging. Particularly, more and more elderly people do not get proper care from individuals and their family anymore. To cope with the situation and overcome the social problems of the elderly population, developed countries in the world aggressively conduct researches and promotions on remote homecare and medical services to solve the problems caused by the elderly population, and also encourage cooperation among related manufacturers, and academic and medical institutes to integrate the current electronic science and technology, network technology and personal medical apparatus and medical care service system and build a remote health care network, in hope of solving the health care issues of elderly people and improve medical services to remote districts with insufficient medical resources.

In general, the so-called “remote homecare” or “remote medical service” connects a patient and a family, or a patient and a doctor at two different places and transmits information such as a patient's physiological parameters (including blood pressure, heartbeat or blood glucose level) to a family or a doctor through Internet or telephone network technology, so as to compile and save the information as the patient's physiological parameter history data and determine the patient's condition. If a patient feels unwell or a patient's life condition is critical, timely homecare or medical services will be provided. Thus, remote homecare or remote medical services can overcome the limitations of time and space by transferring the medical activities originally held at a medical hospital or a clinic to the patient's home.

To achieve the foregoing objective and fully promote the remote homecare or remote medical service, related professionals, hospitals and clinics constantly propose improved technologies and solutions such as the technologies for measuring physiological parameters, sending physiological parameters to a remote end, and expanding the applicable scope of remote homecare, so as to enhance the remote homecare or remote medical service. The technology closely related to users is the technology of transmitting physiological parameters, and a traditional transmission apparatus of physiological parameters transmits the physiological parameters by a physical network cable or a telephone line, and thus the transmission apparatus for transmitting physiological parameters can be installed at a specific position only. As a result, users cannot leave their residence. If a user leaves to a place without installing a transmission apparatus for transmitting physiological parameters at a specific position, the user cannot be monitored and controlled by the remote homecare system anymore, and this is a major drawback of remote homecare.

Although some manufacturers introduced a wireless transmission apparatus for transmitting physiological parameters for users to carry the apparatus with them and eliminate dead corners of monitoring and controlling the remote homecare, the traditional transmission apparatus for transmitting physiological parameters still have the following shortcomings regardless of transmitting physiological parameters by physical cable or wireless:

1. In a traditional transmission apparatus for transmitting physiological parameters, there are several operating steps in the process from the start to the end of transmitting physiological parameters, and only experienced users or professionals can use this apparatus, and such apparatus is not suitable to be used by patients, elderly people or youngsters.

2. Although most traditional transmission apparatuses for transmitting physiological parameters come with a multiple of functions, a user cannot use the transmission apparatus to transmit physiological parameters when there is an emergency. As a result, a medical center, a hospital or a clinic only can base on the received history data of the physiological parameters to determine whether or not the user is in a critical condition, or even miss the prime time for emergency treatments.

If we can use a wireless transmission technology to extend the scope of using the transmission apparatus for transmitting physiological parameters, then users can send the physiological parameters regardless of the location where the user is situated, so as to eliminate the dead corners of the remote homecare. A user-friendly using or operating interface is provided for users to operate the physiological parameter transmission apparatus and transmit the physiological parameters, or a user can issue an emergency notice in an emergency. It is an important subject for related medical centers, hospitals and clinics to study and make further improvements.

In view of the foregoing shortcomings of the traditional transmission apparatus of physiological parameters that is not easy to use or operate and the apparatus has drawbacks since the apparatus can be installed at a certain specific position only, the inventor of the present invention based on years of experience to conduct extensive researches and experiments, and finally invented a wireless transmission apparatus for transmitting physiological parameters and its method in accordance with the present invention to overcome the foregoing shortcomings.

Therefore, it is a primary objective of the present invention to provide a wireless transmission apparatus for transmitting physiological parameters, and the apparatus comprises a processing unit coupled to a connecting unit, a pushbutton, a memory unit and a wireless transmission unit. The connecting unit can be connected to a portable detector outside the wireless transmission apparatus for receiving a physiological parameter of a user measured by the portable detector. The pushbutton is provided for producing a press signal when the pushbutton is pressed, and the memory unit is provided for storing an identification code corresponding to a specific user, such that if the connecting unit is not connected to the portable detector and the processing unit has received the press signal, the processing unit will produce an emergency notice signal and send the emergency notice signal to the wireless transmission unit, and the wireless transmission unit will pack the emergency notice signal and the identification code as an emergency notice packet and will send the emergency notice packet out by a wireless method to a hospital, a clinic or a service provider through a gateway and the Internet. If the connecting unit is connected to the portable detector, and the processing unit has received the press signal, the processing unit will output the physiological parameter to the wireless transmission unit, and the wireless transmission unit will pack the physiological parameter and the identification code as a physiological data packet, and will send the physiological data packet out by a wireless method to a hospital, a clinic or a service provider through a gateway and the Internet for compiling, monitoring and controlling the current physiological conditions of the user.

Another objective of the present invention is to provide a wireless transmission method for transmitting physiological parameters that is applied to a wireless transmission apparatus. If the wireless transmission apparatus is connected to a portable detector, a physiological parameter of a user measured by the portable detector is received, and a press signal is received, then the physiological parameter and an identification code corresponding to a specific user and stored in the wireless transmission apparatus will be packed together as a physiological data packet, and the physiological data packet will be sent to a gateway by a wireless method, and sent to a hospital, a clinic or a service provider through the Internet for compiling, monitoring and controlling the current physiological conditions of the user.

To make it easier for our examiner to understand the objective, technical characteristics and effects of the present invention, a preferred embodiment will be described with accompanying drawings as follows:

### BRIEF DESCRIPTION OF THE DRAWINGS

FIG. 1 is a schematic view of a wireless transmission apparatus in accordance with the present invention;

FIG. 2 is a schematic view of using a wireless transmission apparatus in accordance with the present invention; and

FIG. 3 is a flow chart of the present invention.

### DETAILED DESCRIPTION OF THE PREFERRED EMBODIMENTS

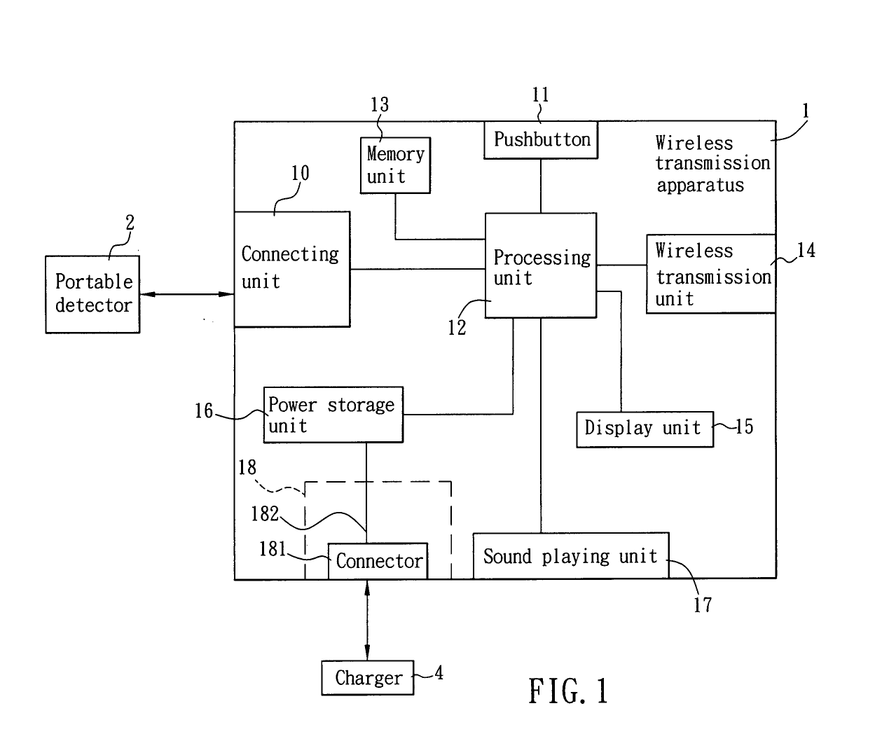

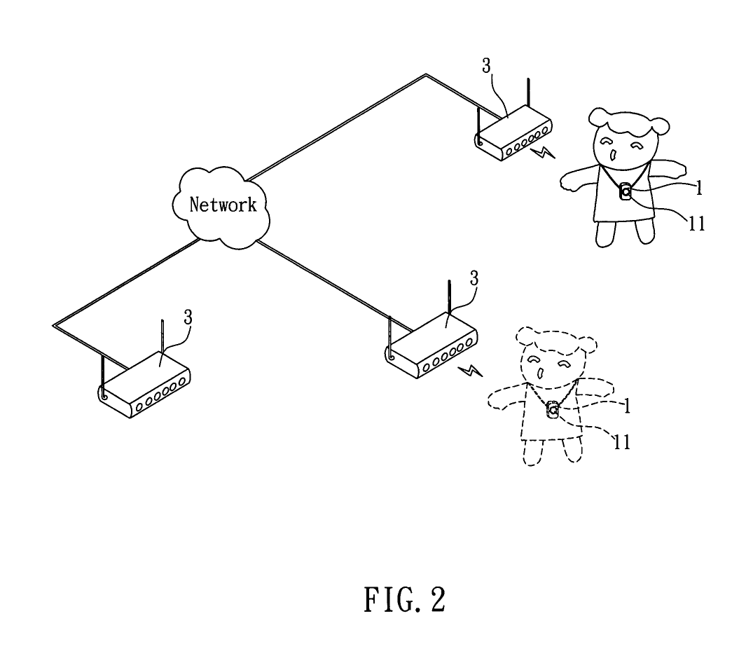

Referring to FIGS. 1 and 2 for schematic views of a wireless transmission apparatus of physiological parameters in accordance with the present invention, the wireless transmission apparatus comprises a connecting unit 10, a pushbutton 11, a processing unit 12, a memory unit 13 and a wireless transmission unit 14. The processing unit 12 is coupled separately to the connecting unit 10, the pushbutton 11, the memory unit 13 and the wireless transmission unit 14. The connecting unit 10 of the wireless transmission apparatus 1 can be connected to a portable detector 2 for receiving a physiological parameter of a user measured by the portable detector 2. The pushbutton 11 will produce a press signal if the pushbutton 11 is pressed. The memory unit 13 is provided for storing an identification code corresponding to a specific user. If the connecting unit 10 is not connected to the portable detector 2, and the processing unit 12 has received the press signal, an emergency of a specific user occurs, and the processing unit 12 will produce an emergency notice signal immediately and send the emergency notice signal to the wireless transmission unit 14, and the wireless transmission unit 14 will pack the emergency notice signal and the identification code together as an emergency notice packet and will send the emergency notice packet out by a wireless method to a hospital, a clinic or a service provider through a gateway 3 and the Internet.

If the connecting unit 10 is connected to the portable detector 2, and the processing unit 12 has received the press signal, the processing unit 12 will send the physiological parameter to the wireless transmission unit 14, and the wireless transmission unit 14 will pack the physiological parameter and the identification code together as a physiological data packet, and will send the physiological data packet out by a wireless method to a hospital, a clinic or a center through a gateway 3 and the Internet for compiling, monitoring and operating the current physiological conditions of the user. Therefore, a user can press the pushbutton 11 to transmit the physiological parameter or emergency notice signal easily that provides a convenient operation. With the wireless transmission unit 14 and gateway 3, a user can monitor and control their physiological conditions regardless of the location of the user, so as to eliminate the dead corners of monitoring and operation of the remote homecare and greatly extend the mobility and scope of activities of a user.

If the emergency notice packet is sent to a server of a hospital, a clinic and a service provider through the gateway 3, the server will search a database of the server based on the identification code carried by the emergency notice packet and will look for a specific user registered in the database and the data of a related contact person of the specific user and will report to the related contact person through a telecommunication network. If the physiological data packet is sent to a server of a hospital, a clinic or a service provider through the gateway 3, the server will search for a database of the server and look for a specific user registered in the database based on the identification code carried by the physiological data packet, and collect and store the physiological parameter of the specific user.

Referring to FIG. 2 for a preferred embodiment of the present invention, the wireless transmission unit 14 can receive a positioning signal outputted by the processing unit 12 after the wireless transmission unit 14 sends the emergency notice packet out, and the wireless transmission unit 14 packs the positioning signal and the identification code as a positioning packet, and sends the positioning packet out by a wireless method. After the positioning packet is sent to a server of a hospital, a clinic or a service provider through the gateway 3 and the Internet, the identification code stored in the positioning packet and the exclusive identification code of the gateway 3 can be used for determining the current location of a user, and thus no matter where the user moves (such as moving to a living room, a garden or a garage), the gateway 3 installed at that particular location can receive the packet transmitted from the wireless transmission apparatus 1, such that the user can be monitored all the time. The invention not only expands the scope of living activities of a user (including home, school, rapid transit station, or park), but also completely eliminates the dead corners of the remote homecare.

Referring to FIG. 1 for a preferred embodiment of the present invention, the surface of a wireless transmission apparatus 1 further includes a display unit 15, such that after the emergency notice packet or the physiological data packet is sent to the outside, the processing unit 12 sends a display signal to the display unit 15 for users to determine from the display of the display unit 15 whether or not the emergency notice packet or the physiological data packet is transmitted to the outside successfully.

In FIG. 1, the wireless transmission apparatus 1 of this embodiment further comprises a power storage unit 16 for storing a stored power supplied for the operation of the wireless transmission apparatus 1, and the wireless transmission apparatus 1 further comprises a sound playing unit 17 coupled to the processing unit 12 for playing a sound signal outputted by the processing unit 12 when the voltage of the stored power is lower than a predetermined value stored in the memory unit 13, so as to remind users about the low level of electric power of the wireless transmission apparatus 1 and prevent the stored power of the wireless transmission apparatus 1 from being exhausted and avoid the situation that users cannot use the wireless transmission apparatus 1.

In FIG. 1, the wireless transmission apparatus 1 of this embodiment further comprises a charging unit 18, and the charging unit 18 includes a connector 181 and a power cable 182. The connector 181 is installed on a surface of the wireless transmission apparatus 1 and coupled to a charger 4 for receiving a charging power supplied by the charger 4, and the power cable 182 is coupled separately to the connector 181 and the power storage unit 16, and the charging power is supplied to the power storage unit 16 through the power cable 182, so as to charge the wireless transmission apparatus 1 and extend the using time of the wireless transmission apparatus 1.

Referring to FIGS. 1 and 2 for a method for transmitting physiological parameters through wireless transmission, the method is used in a wireless transmission apparatus 1. If the wireless transmission apparatus 1 is connected to a portable detector 2, the wireless transmission apparatus 1 will receive a physiological parameter of a user measured by the portable detector 2, and then will determine whether or not a press signal produced by a pushbutton 11 installed on the wireless transmission apparatus 1 is received; if yes, then the physiological parameter and an identification code stored in the wireless transmission apparatus 1 and corresponding to a specific user will be packed as a physiological data packet, and the physiological data packet will be sent to a gateway 3 by a wireless method and then sent to a serve of a hospital, a clinic or a related service provider through the Internet, for compiling, monitoring and controlling the current physiological conditions of a user.

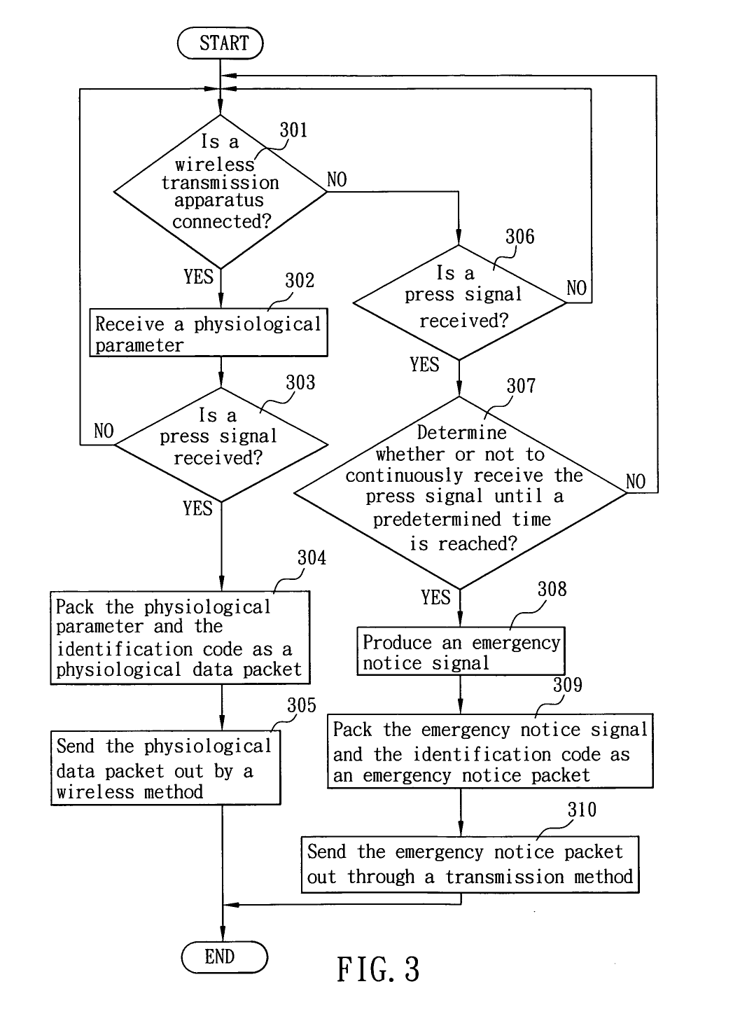

Referring to FIG. 3 for a preferred embodiment of the present invention, the method comprises the steps of:

Step 301: determining whether or not a wireless transmission apparatus 1 is connected to a portable detector 2; if yes, then go to Step 302, or else go to Step 306;

Step 302: receiving a physiological parameter of a user measured by the portable detector 2;

Step 303: determining whether or not a press signal is received; if yes, then go to Step 304, or else go to Step 301;

Step 304: packing the physiological parameter and the identification code as a physiological data packet;

Step 305: sending the physiological data packet out by a wireless method;

Step 306: determining whether or not a press signal is received; if yes, go to Step 307, or else go to Step 301;

Step 307: determining whether or not to continuously receive the press signal until a predetermined time stored in the wireless transmission apparatus 1 is reached; if yes, then go to Step 308, or else go to Step 301;

Step 308: producing an emergency notice signal;

Step 309: packing the emergency notice signal and the identification code as an emergency notice packet; and

Step 310: sending the emergency notice packet out through a transmission method.

If the wireless transmission apparatus 1 is in the mode of receiving physiological parameters, a user can determine whether or not to send the physiological parameters out. If the wireless transmission apparatus 1 has not received a physiological parameter, the user can determine whether or not to send out the emergency notice packet as needed. With the identification code, a server at a hospital, a clinic or a related service provider can find a specific user registered in a database of the serve and the data of a related contact person corresponding to the specific user, when the server receives the emergency notice packet, and then reports to the related contact person through a telecommunication network. When the server receives the physiological data packet, the server finds the specific user registered in the database, and collects and stores the physiological parameter of the corresponding specific user.

Referring to FIG. 2 for a preferred embodiment of the present invention, the emergency notice packet is sent out by a wireless method and the positioning packet is received by the gateway 3 and then sent to a server of a hospital, a clinic or a service provider through the Internet. The identification code stored in the positioning packet and the exclusive identification code of the gateway 3 can be used for determining the current location of a user, and thus no matter where the user moves (such as moving to a living room, a garden or a garage), the gateway 3 installed at that location can receive the packet transmitted from the wireless transmission apparatus 1, such that the user can be monitored all the time. The invention not only expands the scope of living activities of a user (including home, school, rapid transit station, or park), but also completely eliminates the dead corners of the remote homecare.

While the invention herein disclosed has been described by means of specific embodiments, numerous modifications and variations could be made thereto by those skilled in the art without departing from the scope and spirit of the invention set forth in the claims.

1. A wireless transmission apparatus for transmitting physiological parameters, comprising:
a connecting unit, coupled to a portable detector that is located outside said wireless transmission apparatus, for receiving a physiological parameter of a user measured by said portable detector;
a pushbutton, disposed on said wireless transmission apparatus, for producing a press signal when said pushbutton is pressed;
a processing unit, separately coupled to said connecting unit and said pushbutton, such that when said connecting unit is not coupled to said portable detector and said processing unit receives said press signal, said processing unit outputs an emergency notice signal, or when said connecting unit is coupled to said portable detector and said processing unit receives said press signal, said processing unit outputs said physiological parameter;
a memory unit, for storing an identification code, and said identification code corresponding to a specific user; and
a wireless transmission unit, coupled to said processing unit, for packing said emergency notice signal and said identification code as an emergency notice packet and sending said emergency notice packet out by a wireless method when said wireless transmission unit receives said emergency notice signal, or packing said physiological parameter and said identification code as a physiological data packet and sending said physiological data packet out by a wireless method when said wireless transmission unit receives said physiological parameter.

2. The wireless transmission apparatus of physiological parameters of claim 1, wherein a positioning signal and said identification code are packed together as a positioning packet and said positioning packet is sent out by a wireless method, after said emergency notice packet is sent out and said wireless transmission unit receives a positioning signal outputted by said processing unit.

3. The wireless transmission apparatus of physiological parameters of claim 2, further comprising a display unit, for receiving a display signal sent by said processing unit after said emergency notice packet or said physiological data packet is sent out.

4. The wireless transmission apparatus of physiological parameters of claim 3, further comprising a power storage unit, for storing a stored power supplied for the operation of said wireless transmission apparatus.

5. The wireless transmission apparatus of physiological parameters of claim 4, further comprising a sound playing unit, coupled to said processing unit, for playing a sound signal outputted by said processing unit if the voltage of said stored power is lower than a predetermined value stored in said memory unit.

6. The wireless transmission apparatus of physiological parameters of claim 5, further comprising a charging unit, and said charging unit comprising:
a connector, installed on said wireless transmission apparatus and coupled to a charger, for receiving a charging power supplied by said charger; and
a power cable, coupled to said connector and said power storage unit, and said charging power is supplied to said power storage unit through said power cable.

7. A wireless transmission method for physiological parameters, implemented to a wireless transmission apparatus, comprising the steps of: determining whether or not said wireless transmission apparatus is coupled to a portable detector; if it is determined that the wireless transmission apparatus is coupled to a portable detector, then a physiological parameter of a user measured by said portable detector will be received;
determining whether or not a press signal is received; if it is determined that the press signal is received, then said physiological parameter received by said wireless transmission apparatus and an identification code corresponding to a specific user and stored in said wireless transmission apparatus will be packed together as a physiological data packet; and
sending said physiological data packet out to the Internet by a wireless method.

8. The wireless transmission method for physiological parameters of claim 7, further comprising a procedure when said wireless transmission apparatus is not coupled to said portable detector, said procedure comprising the steps of:
determining whether or not said press signal is received; if it is determined that the press signal is received, then an emergency notice signal will be produced;
packing said emergency notice signal and said identification code together as an emergency notice packet; and
sending said emergency notice packet out by a wireless method.

9. The wireless transmission method for physiological parameters of claim 8, further comprising a procedure after said press signal is received and before said emergency notice signal is produced, and said procedure comprising the steps of:
determine whether or not to continue receiving said press signal until a predetermined time stored in said wireless transmission apparatus is reached; if it is determined that the predetermined time stored in said wireless transmission apparatus is reached, said emergency notice signal will be produced.

10. The wireless transmission method for physiological parameters of claim 8, further comprising a step of sending a positioning packet out periodically by a wireless method, after said emergency notice packet is sent out by a wireless method.
